(19) United States
(12) Patent Application Publication
(10) Pub. No.: US 2022/0409170 A1
(43) Pub. Date: Dec. 29, 2022

(54) ULTRASOUND TRANSDUCER

(71) Applicant: Boston Scientific Scimed, Inc., Maple Grove, MN (US)

(72) Inventors: Andrew GRAVELEY, Indianapolis, IN (US); Lawrence BACKMAN, Shoreview, MN (US)

(73) Assignee: Boston Scientific Scimed, Inc., Maple Grove, MN (US)

(21) Appl. No.: 17/806,776

(22) Filed: Jun. 14, 2022

Related U.S. Application Data
(60) Provisional application No. 63/202,764, filed on Jun. 23, 2021.

(51) Int. Cl. A61B 8/12; A61B 8/00

(52) U.S. Cl. CPC A61B 8/12; A61B 8/445; A61B 8/4483

(57) ABSTRACT

A transducer device may include an active layer having a proximal surface and a backing layer having a distal side and a proximal side, the distal side being adjacent to the proximal surface. The proximal side may include (1) at least one first reflective surface approximately parallel to the proximal surface and positioned a first distance from the proximal surface, and (2) at least one second reflective surface approximately parallel to the proximal surface and positioned a second distance from the proximal surface, the second distance being different than the first distance.

### CROSS-REFERENCE TO RELATED APPLICATIONS

This application claims the benefit of priority of U.S. Provisional Patent Application No. 63/202,764, filed Jun. 23, 2021, the entirety of which is incorporated herein by reference.

### TECHNICAL FIELD

Various aspects of this disclosure relate generally to transducer devices. More specifically, the disclosure relates to transducer devices used in minimally invasive ultrasound imaging systems.

### BACKGROUND

As ultrasound imaging devices continue to incorporate more transducers to produce higher quality ultrasound images, the overall size of ultrasound imaging devices remains limited by the patient anatomy accessed and imaged by them. Accordingly, there is an increased demand for smaller transducers to minimize invasiveness while accessing patient anatomy. A conventional transducer, however, cannot simply be made smaller by making its constituent elements smaller without impacting performance of the transducer and in turn negatively affecting the quality of ultrasound images produced.

### SUMMARY

Aspects of the disclosure relate to, among other things, transducer devices and ultrasound imaging systems incorporating transducer devices.

According to an example, a transducer device may include an active layer having a proximal surface; and a backing layer having a distal side and a proximal side, the distal side being adjacent to the proximal surface. The proximal side may include (1) at least one first reflective surface approximately parallel to the proximal surface and positioned a first distance from the proximal surface, and (2) at least one second reflective surface approximately parallel to the proximal surface and positioned a second distance from the proximal surface, the second distance being different than the first distance. The at least one first reflective surface may have a first area, and the at least one second reflective surface may have a second area approximately equal to the first area, and the sum of the first area and the second area may be approximately equal to an area of all surfaces of the proximal side that are approximately parallel to the proximal surface.

According to another example, a transducer device may include an active layer having a proximal surface; and a backing layer having a distal side and a proximal side, the distal side being adjacent to the proximal surface. The proximal side of the backing layer may include (1) one or more first reflective surfaces and (2) one or more second reflective surfaces, the first and second reflective surfaces being approximately parallel to the proximal surface. The first reflective surfaces may be recessed a first distance from the second reflective surfaces; and the first reflective surfaces and the second reflective surfaces may be the only surfaces included on the proximal side that are approximately parallel to the proximal surface, and a surface area of the first reflective surfaces is approximately equal to a surface area of the second reflective surfaces.

According to another example, an ultrasound imaging system may include a catheter configured and arranged for insertion into a living being, the catheter having a distal end and a proximal end; and an imaging device on the catheter, the imaging device configured for imaging interior portions of the living being. The imaging device may include a plurality of transducer devices. Each of the transducer devices may include an active layer having an proximal surface; and a backing layer having a distal side and a proximal side, the distal side being adjacent to the proximal surface. The proximal side may include (1) at least one first reflective surface approximately parallel to the proximal surface and positioned a first distance from the proximal surface, and (2) at least one second reflective surface approximately parallel to the proximal surface and positioned a second distance from the proximal surface, the second distance being different than the first distance. The at least one first reflective surface may have a first area, and the at least one second reflective surface may have a second area approximately equal to the first area, and the sum of the first area and the second area may be approximately equal to an area of all surfaces of the proximal side that are approximately parallel to the proximal surface.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 is a perspective view of an ultrasound imaging device, according to aspects of this disclosure;

FIG. 2 is a perspective view of a distal end of a catheter of an ultrasound imaging device, according to aspects of this disclosure;

FIG. 3 is a side view of a transducer device, according to aspects of this disclosure;

FIG. 4 is a graphical representation of a signal generated by a transducer device, according to aspects of this disclosure;

FIG. 5A is a side view of an active layer, according to aspects of this disclosure;

FIG. 5B is a side view of an active layer, according to aspects of this disclosure;

FIG. 6 is a perspective view of a transducer device, according to aspects of this disclosure;

FIG. 7 is a side view of a transducer device, according to aspects of this disclosure;

FIG. 8A is a partial cross-sectional view of a transducer device, according to aspects of this disclosure;

FIG. 8B is a partial cross-sectional view of a transducer device, according to aspects of this disclosure;

FIG. 9A is a graphical representation of reflected acoustic energy, according to aspects of this disclosure;

FIG. 9B is a graphical representation of reflected acoustic energy, according to aspects of this disclosure.

### DETAILED DESCRIPTION

This disclosure relates to transducer devices and ultrasound imaging devices incorporating transducer devices. Specifically, transducer devices having backing layers configured to reduce an effect of acoustic energy reflected back to an active layer are described herein. By reducing the effect of reflected acoustic energy on a signal generated by the transducer device, a thickness of the backing layer may be reduced without impacting a quality of an image produced. In turn, an overall size of the transducer device may be minimized. Accordingly, the size of an ultrasound image device incorporating transducer devices may also be reduced, minimizing invasiveness of procedures in which an ultrasound imaging device is used. Alternatively, or in addition, more transducer devices may be incorporated in an ultrasound imaging device without significantly increasing the size of the ultrasound imaging device, thereby improving the image generated by the device.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−5% of a stated value.

Examples of the disclosure may relate to transducer devices and ultrasound imaging devices incorporating transducer devices. This disclosure is not limited to any specific medical device or method, however, and aspects of the disclosure may be used in connection with any suitable medical tool and/or medical method, at any suitable site within the body. For example, transducer devices according to this disclosure may be incorporated within therapeutic ultrasound devices. Various examples described may include single-use or disposable medical devices.

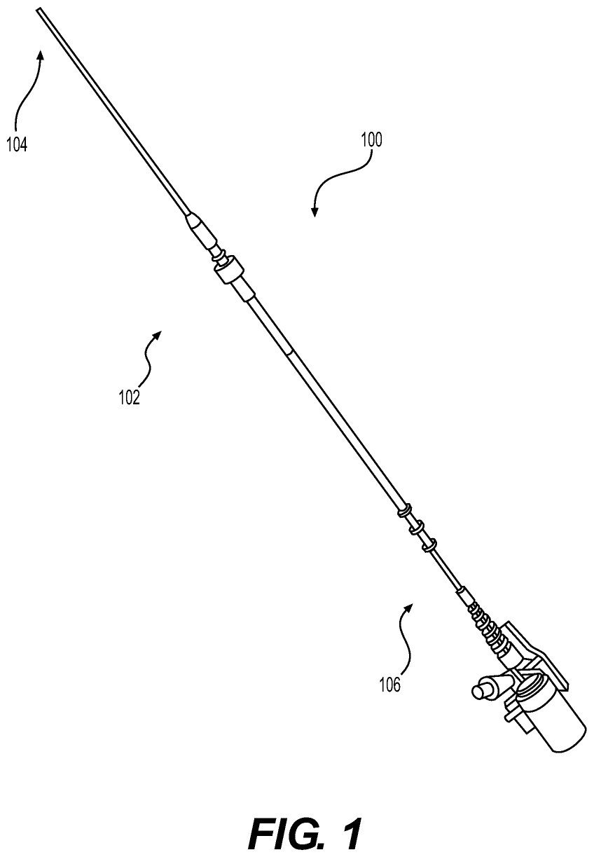

FIG. 1 illustrates an exemplary ultrasound imaging device 100 in accordance with one or more examples of this disclosure. Ultrasound imaging device 100 may be configured to be inserted into a patient for imaging of portions of the patient's anatomy. Ultrasound imaging device 100 may be, for example, an intra-cardiac echocardiography (ICE) device, an endobronchial ultrasound (EBUS) device, an intra-vascular ultrasound (IVUS) device, or any other type of ultrasound imaging device. Ultrasound imaging device 100 may include a catheter 102 having a proximal end 106 and a distal end 104. The proximal end 106 of the catheter 102 may be coupled to a catheter hub and the distal end 104 may be configured and arranged for insertion into a patient. Catheter 102 may be dimensioned such that it can be inserted within regions of a patient that are difficult to navigate, including blood vessels, heart chambers, the gastrointestinal tract, the urinary tract, the pulmonary system, or the like.

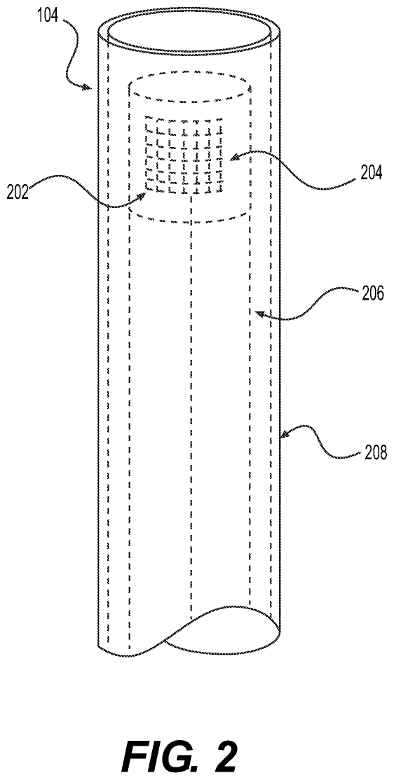

Distal end 104 of catheter 102 may include one or more transducer devices positioned therein. FIG. 2 is a perspective view of distal end 104 of catheter 102 according to some embodiments. Catheter 102 may include a sheath 208 defining a lumen and surrounding an imaging core 206 disposed in the lumen. The imaging core 206 may include an imaging module 202 disposed in the catheter 102. One or more transducer devices 204 may be included in imaging module 202 and may be used to transmit and receive acoustic energy to generate ultrasound images. The sheath 208 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In some embodiments, and as shown in FIG. 2, an array of a plurality of transducer devices 204 may be included in imaging module 202. In some embodiments, an application-specific integrated circuit (ASIC) associated with the transducer devices 204 may be positioned behind or underneath the array. In some embodiments, a single transducer device 204 may be employed. Any suitable number of transducer devices 204 may be used. For example, there may be two, three, four, five, six, seven, eight, nine, ten, twelve, fifteen, sixteen, twenty, twenty-five, fifty, one hundred, five hundred, one thousand, or more transducer devices 204. As will be recognized, other numbers of transducer devices 204 may also be used. When a plurality of transducer devices 204 are employed, the transducer devices 204 may be arranged in any suitable manner including, for example, an annular arrangement, a rectangular arrangement, or the like.

In order to incorporate a greater quantity of transducer devices 204 while maintaining the dimensions of catheter 102 such that catheter 102 may be inserted into regions of the body such as those discussed herein, it may be desirable to reduce the size of the transducer devices 204. Reducing the size of traditional transducer devices generally cannot be accomplished without significant sacrifices in performance. Further reduction of a size of conventional transducer devices may therefore be practically limited.

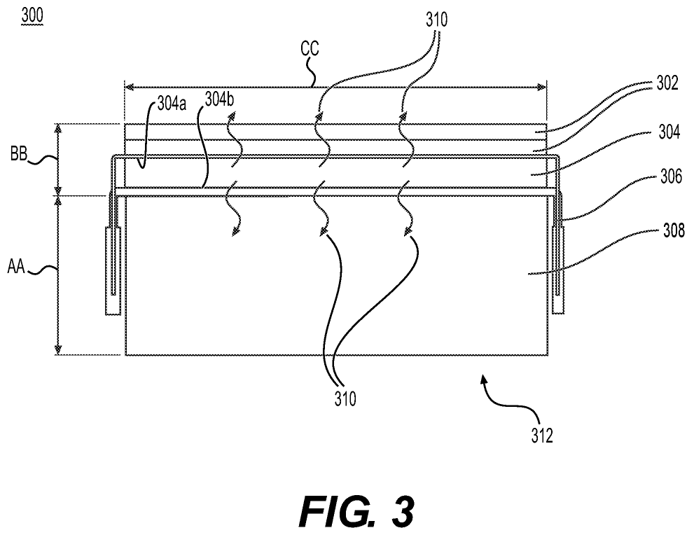

FIG. 3 illustrates an example of a transducer device 300, the limitations of which are described herein. Transducer device 300 may include one or more matching layers 302, an active layer 304, electrical connecters 306, and backing layer 308. In some embodiments, matching layers 302 may be omitted. Active layer 304 may be formed from one or more known materials capable of transforming applied electrical signals to acoustic energy emanating from the surface of the active layer 304, and conversely capable of transforming acoustic energy absorbed by the active layer 304 into electrical signals. Examples of suitable materials may include piezoelectric ceramic materials, piezocomposite materials, piezoelectric plastics, barium titanates, lead zirconate titanates, lead metaniobates, polyvinylidenefluorides, and the like. Other transducer technologies may include composite materials, single-crystal composites, and semiconductor devices, such as capacitive micromachined ultrasound transducers (“cMUT”), piezoelectric micromachined ultrasound transducers (“pMUT”), or the like. Active layer 304 may include a distal surface 304a and a proximal surface 304b. FIG. 3 depicts acoustic energy 310 emanating from both distal surface 304a and proximal surface 304b of active layer 304.

Electrical signals may be applied to the active layer 304 via electrical connecters 306. For example, a voltage may be applied to active layer 304 via electrical connecters 306 to generate a pulse of acoustic energy that emanates from active layer 304 toward an imaging target. The pulse of acoustic energy may include one or more acoustic waves and may have an associated pulse duration and pulse length. When the pulse of acoustic energy reflects off the imaging target and the reflection is received by active layer 304, active layer 304 may generate an electrical signal that may be measured and/or detected via electrical connecters 306. The electrical signal may in turn be used to generate an ultrasound image of the target.

The one or more matching layers 302 may be acoustically coupled to a distal side of active layer 304 and may be provided in order to facilitate transfer of acoustic energy from the active layer 304 to a propagating medium, such as ultrasound gel, by reducing losses due to reflection of the acoustic energy. Matching layers 302 may have a thickness and acoustic impedance such that acoustic energy reflected back toward the active layer 304 from the interfaces between active layer 304 and matching layers 302, or matching layers 302 and the propagating medium are minimized. The thickness of each matching layer 302 may be determined based on a central frequency acoustic energy emitted by the transducer device 300 and the speed of sound of the material. Matching layers 302 may facilitate the transfer of acoustic energy from the active layer 304 to the propagating medium (and vice versa) within a bandwidth of frequencies around the center frequency of the device.

Backing layer 308 may be positioned adjacent to proximal surface 304b of active layer 304. Backing layer 308 may be formed from one or more known materials suitable for absorbing, scattering, and/or attenuating acoustic energy, including polymers, composite materials incorporating metallic scattering particles and/or ceramic oxide scattering particles, and/or viscoelastic materials. Backing layer 308 may dampen vibration of active layer 304 so as to control the pulse length and pulse duration. For example, when an electrical signal is applied to active layer 304 and transducer device 300 is fired, active layer 304 may vibrate. Absent backing layer 308 for dampening, active layer 304 would continue to vibrate for a significant duration after the electrical signal is no longer applied. When positioned adjacent to the proximal surface 304b of active layer 304, backing layer 308 may absorb, scatter, and/or attenuate acoustic energy 310 emitted from active layer 304, thereby allowing active layer 304 to cease vibrating rapidly when application of the electrical signal ceases.

As shown in FIG. 3, a cross-sectional size of transducer device 300 may depend on thickness AA of backing layer 308, thickness BB of active layer 304, the one or more matching layers 302, and electrical connectors 306, and width CC of active layer 304. Generally, thickness AA may be larger than thickness BB. In many cases, reducing the overall size of transducer device 300 by reducing any of these dimensions may not be feasible. For example, reducing width CC may sacrifice sensitivity and beam focus of the transducer device 300 because reduction in the surface area of active layer 304 may result in less acoustic energy produced and reduction of the elevation width of active layer 304 may result in a more divergent beam.

Thickness AA of backing layer 308 may be a thickness sufficient to adequately absorb, scatter, and/or attenuate the acoustic energy emanated from or passing through active layer 304. Reducing thickness AA may result in significant negative impacts to image quality due to insufficient attenuation of acoustic energy. For example, if thickness AA is not sufficient to adequately attenuate acoustic energy emanated from or passing through active layer 304, the acoustic energy may reflect off proximal side 312 of backing layer 308 and back toward active layer 304. As a result, a pulse length of the acoustic energy may be effectively increased and the axial resolution of transducer device 300 effectively reduced.

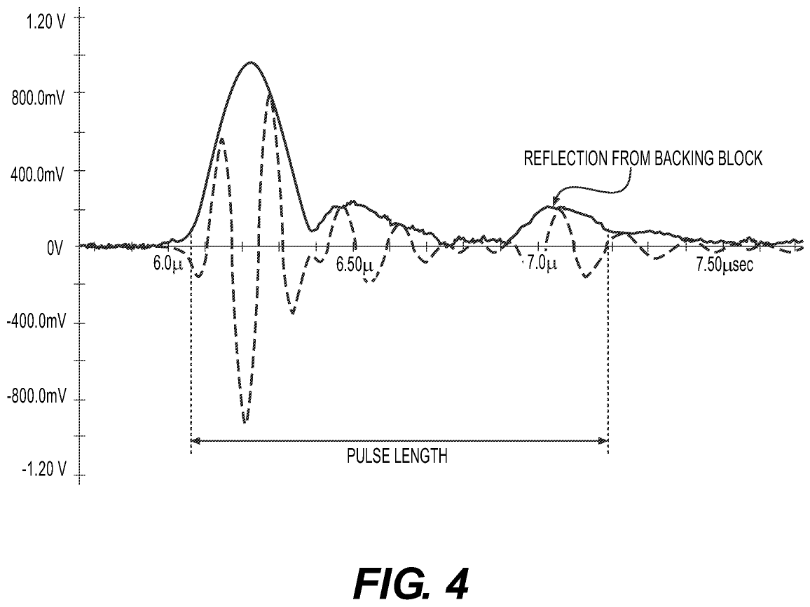

FIG. 4 depicts a graphical representation of a pulse of acoustic energy emitted from active layer 304 when thickness AA of backing layer 308 is insufficient to adequately attenuate the acoustic energy traveling from the proximal surface 304b of active layer 304 to proximal side 312. As shown, from about 6.0 psec to about 6.4 psec, active layer 304 may be fired and may emit acoustic energy in the form of acoustic waves. From about 6.4 psec to 6.9 psec, firing of active layer 304 may cease and active layer 304 may be dampened by backing layer 308. At about 7.0 psec, however, acoustic energy emitted from the proximal surface 304b of active layer 304 and reflected off of proximal side 312 of backing layer 308 may travel back to active layer 304. The reflected acoustic energy may appear as an artifact in the electrical signal produced by the active layer 304. This artifact from the reflected acoustic energy may effectively increase the pulse length thereby reducing the axial resolution of transducer device 300. In other words, in a 2D ultrasound image generated from the electrical signal transmitted by transducer device 300, the artifact may appear as a blur near any tissue or material boundary in the image.

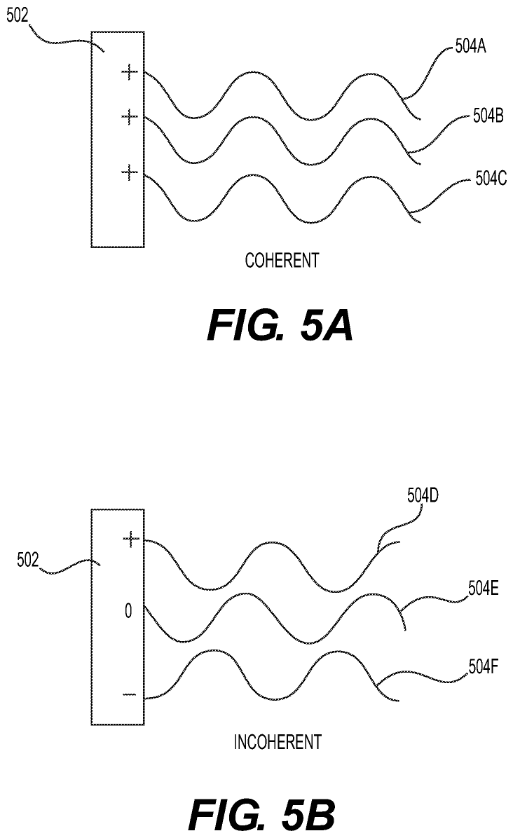

Accordingly, thickness AA may not simply be reduced without significantly reducing performance of transducer device 300. The artifact shown in FIG. 4, however, may be produced when acoustic waves reflected off of proximal side 312 of backing layer 308 are coherent, or in phase. FIG. 5A depicts an effect of a plurality of coherent acoustic waves 504A-C passing into an active layer 502. When coherent acoustic waves 504A-C reach active layer 502, like voltages produced by each individual wave at active layer 502 may accumulate on active layer 502 and create a large electrical signal that changes approximately uniformly across active layer 502 with the oscillating pressure of the acoustic waves 504A-C.

FIG. 5B depicts a plurality of incoherent acoustic waves 504D-E passing into active layer 502. In contrast with waves 504A-C, incoherent acoustic waves 504D-E are out of phase with each other. When incoherent acoustic waves 504D-E reach active layer 502, random voltages produced by each individual wave at active layer 502 may accumulate on active layer 502, thereby creating a cumulative charge that may be approximately zero. Therefore, a discernible electrical signal may not be generated by active layer 502 in response to incoherent acoustic waves 504D-E.

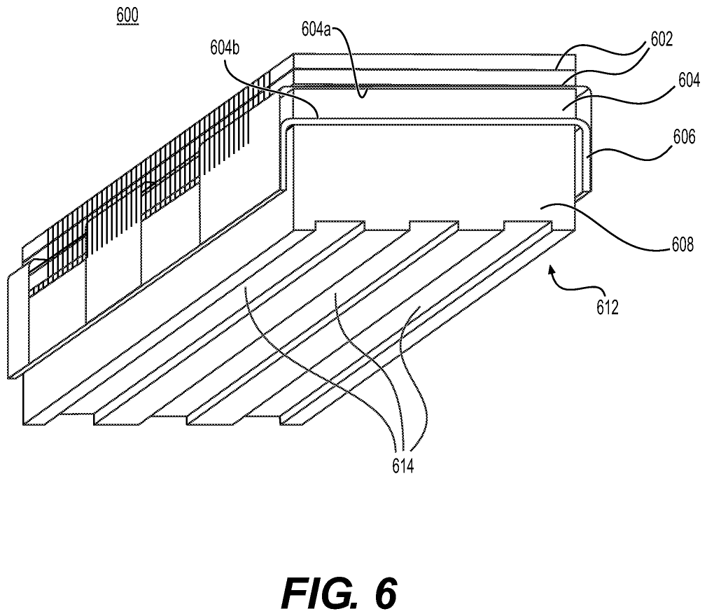

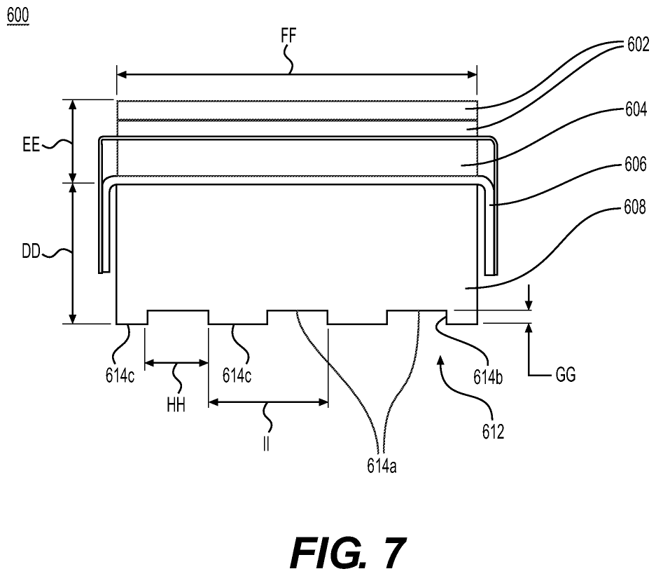

Applying the phenomenon depicted in FIG. 5B to reflected acoustic energy in a transducer device, a size of the transducer device may be effectively reduced without significant impact to performance of the transducer device. FIGS. 6 and 7 depict an exemplary transducer device 600 having a backing layer with a reduced thickness. Similar to transducer device 300 described herein previously, transducer device 600 may include one or more matching layers 602, an active layer 604, and electrical connecters 606. Similar to active layer 304, active layer 604 may be formed from one or more known materials capable of transforming applied electrical signals to acoustic energy and of transforming acoustic energy absorbed by the active layer 604 into electrical signals. Active layer 604 may include a distal surface 604a and a proximal surface 604b.

Electrical signals may be applied to the active layer 604 via electrical connecters 606. For example, a voltage may be applied to active layer 604 via electrical connecters 606 to generate a pulse of acoustic energy that emanates from active layer 604 toward an imaging target. The pulse of acoustic energy may include one or more acoustic waves and may have an associated pulse duration. When the pulse of acoustic energy reflects off the imaging target and is received by active layer 604, active layer 604 may generate an electrical signal that can be measured and/or detected via electrical connecters 606.

Exemplary transducer device 600 may further include a backing layer 608 positioned adjacent to a proximal surface 604b of active layer 604. Backing layer 608 may dampen vibration of active layer 604 so as to control the pulse length and pulse duration. For example, when transducer device 600 is fired and an electrical signal is applied to active layer 604, active layer 604 may vibrate. When positioned adjacent to the proximal surface 604b of active layer 604, backing layer 608 may absorb, scatter, and/or attenuate acoustic energy emanated from active layer 604, thereby allowing active layer 604 to cease vibrating rapidly when application of the electrical signal is ceased.

Backing layer 608 may include a proximal side 612 in which a plurality of grooves 614 are formed. The grooves 614 may be formed using a dicing saw, for example, or by any other suitable method. In some embodiments, and as shown in FIG. 6, grooves 614 may extend along an entire length (the larger dimension) of proximal side 612. In some embodiments, grooves 614 may extend along an entire width (the shorter dimension) of proximal side 612. In other embodiments, grooves 614 may be oriented at an angle relative to the length of proximal side 612, such as 15 degrees, 30 degrees, 45 degrees, 60 degrees, 75 degrees, or any other angle. Each of the grooves 614 may be defined by an internal surface 614a and at least one side surface 614b. Each internal surface 614a may be approximately parallel to proximal surface 604b of active layer 604. Each side surface 614b may be approximately perpendicular to proximal surface 604b of active layer 604. In some embodiments, side surfaces 614b may be angled and/or have rounded edges. Angling of side surfaces 614b and/or incorporating rounded edges therewith may increase scattering of emitted acoustic energy. External surfaces 614c may be defined by portions of proximal side 612 in which a groove 614 is not located and may be adjacent to one or more grooves 614. External surfaces 614c may likewise be approximately parallel to proximal surface 604b.

Internal surfaces 614a may have a cumulative surface area that is approximately equal to a cumulative surface area of external surfaces 614c. In some embodiments, internal surfaces 614a and external surfaces 614c may extend substantially across an entirety of proximal side 612 of backing layer 608. In other words, a sum of the areas of internal surfaces 614a and the areas of external surfaces 614c may be approximately equal to a total surface area of proximal side 612.

As shown in FIG. 7, backing layer 608 may have a thickness DD, active layer 604 and one or more matching layers 602 may have a cumulative thickness EE, and active layer 604 may have a width FF. Thickness DD may be significantly smaller than thickness AA of backing layer 308 depicted in FIG. 3. Thickness DD may further be less than a thickness sufficient to adequately attenuate acoustic energy emitted from active layer 604 toward proximal side 612. In other words, thickness DD may be a value such that acoustic energy emitted from active layer 604 may reflect off proximal side 612 and travel back to active layer 604.

Internal surfaces 614a may have a width HH. Each internal surface 614a and an adjacent external surface 614c may have a combined width II. In some embodiments, the width HH of each internal surface 614a may be equal to a width of each external surface 614c such that width II is twice width HH. In some embodiments, the widths of each internal surface 614a may be equal to each other. In some embodiments, the widths of each external surface 614c may be equal to each other. In some embodiments, the widths of each internal surface 614a and external surface 614c may be equal to each other.

Each of the grooves 614 may further have a depth GG. Depth GG may be determined based on a wavelength of acoustic waves that reflect off of proximal side 612 back to active layer 604. The wavelength (λ) of acoustic waves may be calculated according to the following formula, where c represents a longitudinal sound velocity of backing layer 608 and f_{R} represents a center frequency of the reflection from proximal side 612:

$$λ = \frac{c}{{f}_{R}}$$

In the equation above, longitudinal sound velocity c may vary depending on the material from which backing layer 608 is formed. Further, center frequency f_{R} of the acoustic waves reflected off of proximal side 612 may differ from a center frequency of the acoustic energy emitted from active layer 604 because attenuation by backing layer 608 may remove a proportionally greater amount of high frequency acoustic energy.

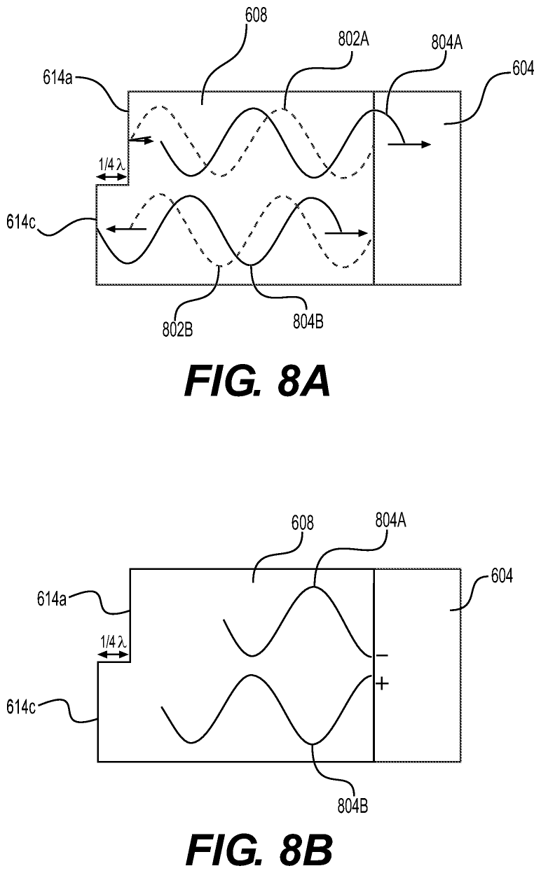

In order to reduce an impact of acoustic energy reflected from proximal side 612 on the electrical signal generated by active layer 604, depth GG may be about one quarter wavelength (λ) of the center frequency of the acoustic energy reflected off of proximal side 612. FIGS. 8A and 8B depict partial cross-sectional views of transducer device 600 and illustrate an effect of incorporating grooves 614 having a depth of about one quarter wavelength (λ).

As shown in FIG. 8A, active layer 604 may emit acoustic waves 802A and 802B each having a wavelength (λ). Acoustic waves 802A and 802B may be in phase upon emission from active layer 604. Acoustic waves 802A and 802B may travel through backing layer 608 and reflect off of internal surface 614a and external surface 614c, respectively, to create reflected waves 804A and 804B. Reflected waves 804A and 804B may also have the same wavelength (λ) and may travel through backing layer 608 back toward active layer 604.

Internal surface 614a and external surface 614c may be offset by one quarter of the wavelength (λ). Due to the offset, acoustic wave 802B may travel one quarter wavelength further than acoustic wave 802A before reflecting. Also due to the offset, reflected wave 804B may travel one quarter wavelength further than reflected wave 804A before reaching active layer 604. Accordingly, as shown in FIG. 8B, when reflected waves 804A and 804B reach active layer 604 after traveling through backing layer 608, reflected wave 804B may be one half wavelength out of phase with reflected wave 804A. Reflected wave 804A and reflected wave 804B may therefore generate opposite charges on active layer 604, effectively cancelling each other out in the electrical signal generated by active layer 604.

While FIGS. 8A and 8B depict waves 802A, 802B, 804A, and 804B, each having a single wavelength (λ) for simplicity, it is to be understood that active layer 604 may emit acoustic waves having other wavelengths, some of which may be attenuated by backing layer 608 before reaching internal surface 614a or external surface 614c. The remaining acoustic waves that are not attenuated may reflect off of internal surface 614a or external surface 614c such that the total reflected acoustic energy may have an associated center frequency. The offset between internal surface 614a and external surface 614c may be one quarter of a wavelength corresponding to the center frequency such that a maximum amount of acoustic energy is cancelled out by the offset.

Additionally, in some embodiments, the offset may be an odd multiple of one quarter wavelength corresponding to the central frequency. For example, the offset may be three-fourths of a wavelength, five-fourths of a wavelength, seven-fourths of a wavelength, or any other odd multiple of one quarter wavelength. By incorporating any offset being an odd multiple of one quarter wavelength, waves reflected off of internal surfaces 614a and external surfaces 614b may be one half wavelength out of phase when they reach active layer 604.

Referring back to FIGS. 6 and 7, by including grooves 614 having a depth of about one quarter wavelength corresponding to the center frequency of the acoustic waves reflected from proximal side 612, acoustic energy reflecting off of internal surfaces 614a may effectively cancel a portion of the acoustic energy reflecting off of external surfaces 614c. Furthermore, if internal surfaces 614a have a cumulative surface area that is approximately equal to a cumulative surface area of external surfaces 614c, acoustic energy reflecting off of internal surfaces 614a may effectively cancel a maximum amount of acoustic energy reflecting off of external surface 614c. Accordingly, thickness DD of backing layer 608 may be reduced significantly from a thickness required to sufficiently attenuate the acoustic energy emitted by active layer 604 without allowing reflection off of proximal side 612. Consequently, an overall size of exemplary transducer device 600 may be reduced without significantly sacrificing performance.

While proximal side 612 is shown in FIGS. 6 and 7 as having grooves 614, the configuration shown is exemplary and other configurations are possible. For example, proximal side 612 may include grooves, circular recesses, square recesses, triangular recesses, any other shape of recess, or any combination of the foregoing. In some embodiments, approximately half of proximal side 612 may be recessed, without limitation as to the shape or position of any recesses. In some embodiments, proximal side 612 may include only two levels of surfaces: one level that is recessed and another level that is not recessed. In some embodiments, the recessed level may be a distance approximately equal to one quarter wavelength from the level that is not recessed. In some embodiments, the recessed level may be a distance approximately equal to an odd multiple of one quarter wavelength from the level that is not recessed.

In some embodiments, for further management of reflected acoustic energy in wide-bandwidth transducer devices, a backing layer including grooves may be configured to attenuate higher frequencies with absorption and scattering, and the grooves may be cut to a depth of about one quarter wavelength of a lower target frequency. Such a backing layer may effectively minimize the impact of both higher and lower frequency reflected acoustic energy.

Moreover, manufacturing of exemplary transducer device 600 having grooves 614 may require minimal additional manufacturing steps. For example, transducer device 600 may be manufactured using conventional steps with an additional step of cutting grooves 614 into proximal side 612 using a dicing saw. Incorporating grooves 614 extending across substantially an entire length of 612 may also maximize a structural rigidity of exemplary transducer device 600.

Furthermore, testing of the subject matter described herein was performed using a prototypical backing layer, the results of which demonstrated the effectiveness of the aforementioned configurations. For the testing, a sample of EpoTek 301-2 Epoxy was machined into a flat disk. Initially, the sample did not include grooves. Acoustic energy may experience a low level of attenuation when traveling through EpoTek 301-2 Epoxy, so reflections within the sample were expected to retain a large portion of the emitted acoustic energy.

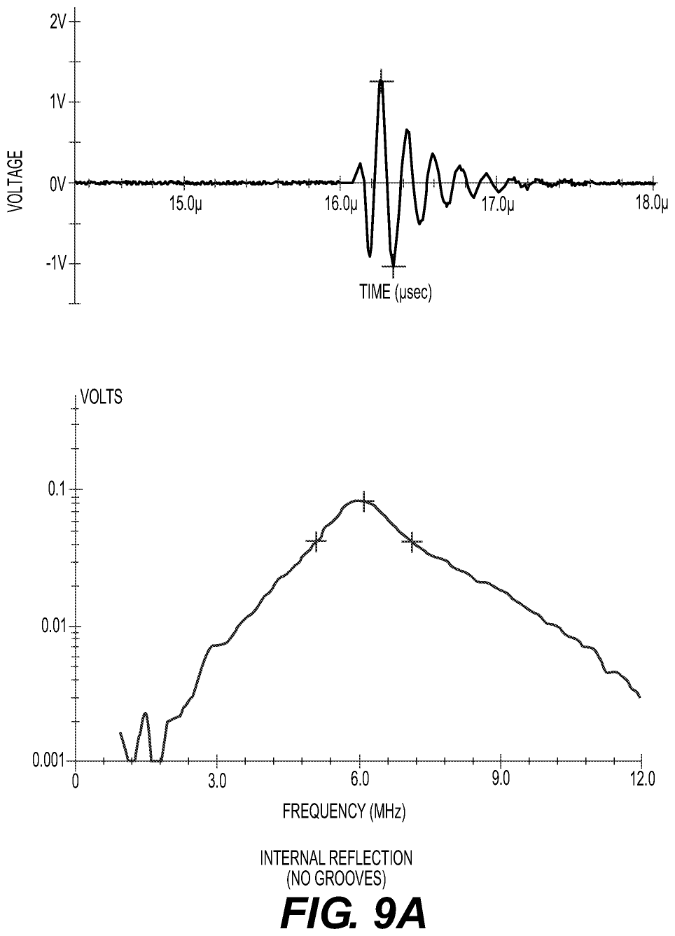

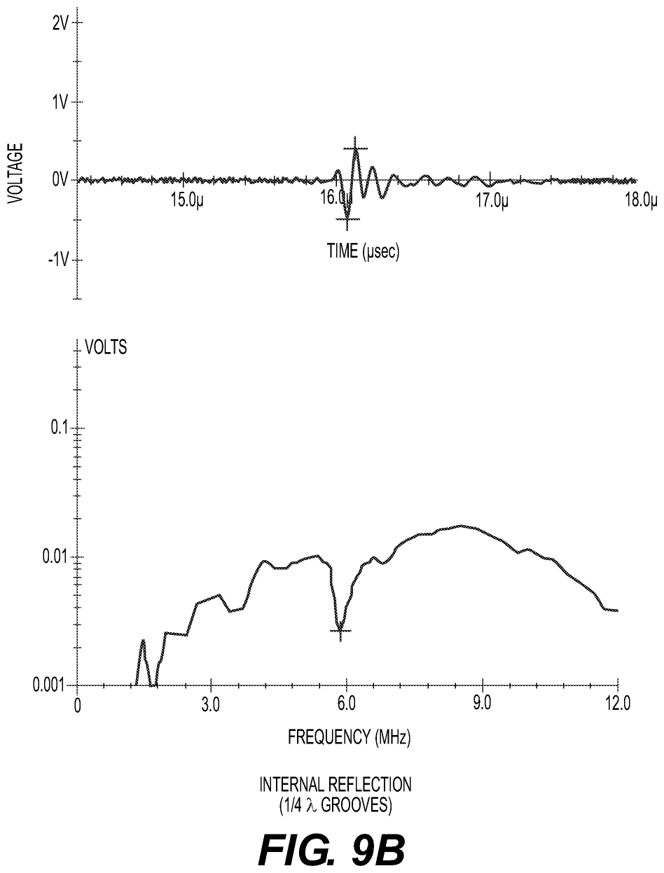

The sample was first pulse-echo tested with a large-aperture single element transducer. FIG. 9A is a graphical illustration of internal reflections of the acoustic energy transmitted into the sample by the transducer. The internal reflections had a center frequency of approximately 5.9 MHz. An acoustic velocity of the epoxy was measured to be approximately 2700 m/s.

For a subsequent test, grooves were cut into the sample. Based on the 5.9 MHz center frequency and the 2700 m/s acoustic velocity, a wavelength corresponding to the center frequency was determined to be about 456 μm. Accordingly, grooves of about 114 μm (corresponding to one quarter of the wavelength) were cut into the proximal side of the sample. The grooves were cut so as to occupy approximately one half of the surface area of the proximal side of the sample.

The sample including grooves was then pulse-echo tested again under the same conditions, to evaluate the design. FIG. 9B is a graphical illustration of the internal reflections of the acoustic energy transmitted to the sample by the transducer. FIG. 9B demonstrates that the addition of the grooves to the sample, without any other changes, reduced the 5.9 MHz target frequency by approximately 30 dB. The one quarter wavelength grooves may therefore effectively mitigate the effect of internal reflection of acoustic energy at a targeted frequency.

Each of the aforementioned systems, devices, and assemblies may allow a size of an ultrasound imaging device to be minimized without sacrificing performance. By configuring a backing layer of a transducer device to minimize the effect of acoustic energy reflected off of a proximal side of the backing layer, a thickness of the backing layer may be reduced without significantly impacting a quality of an image produced by the transducer device. An overall size of the transducer device may in turn be reduced and a size of an ultrasound imaging device into which the transducer device is incorporated may be minimized. Consequently, the invasiveness of medical procedures utilizing the imaging device may likewise be minimized.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A transducer device comprising:
an active layer having a proximal surface; and
a backing layer having a distal side and a proximal side, the distal side being adjacent to the proximal surface;
wherein the proximal side includes (1) at least one first reflective surface approximately parallel to the proximal surface and positioned a first distance from the proximal surface, and (2) at least one second reflective surface approximately parallel to the proximal surface and positioned a second distance from the proximal surface, the second distance being different than the first distance;
wherein the at least one first reflective surface has a first area, and the at least one second reflective surface has a second area approximately equal to the first area, and the sum of the first area and the second area is approximately equal to an area of all surfaces of the proximal side that are approximately parallel to the proximal surface.

2. The transducer device of claim 1, wherein the at least one first reflective surface and the at least one second reflective surface are the only surfaces included on the proximal side that are approximately parallel to the proximal surface.

3. The transducer device of claim 1, wherein the proximal side of the backing layer includes a plurality of grooves, each of the plurality of grooves being adjacent to at least one first reflective surface.

4. The transducer device of claim 1, wherein the active layer includes a piezoelectric material that emits acoustic waves in response to application of an electric signal to the active layer.

5. The transducer device of claim 4, wherein the first distance and the second distance are such that acoustic waves reflected from the at least one first reflective surface are approximately one half wavelength out of phase with acoustic waves reflected from the at least one second reflective surface.

6. The transducer device of claim 5, wherein a cumulative charge generated by the active layer in response to the acoustic waves reflected from the at least one first reflective surface and the at least one second reflective surface is approximately zero.

7. The transducer device of claim 4, wherein a difference between the first distance and the second distance is approximately one quarter of a wavelength of the acoustic waves.

8. The transducer device of claim 4, wherein a difference between the first distance and the second distance is approximately one quarter of a wavelength corresponding to a center frequency of acoustic waves that reflect off of the at least one first reflective surface and the at least one second reflective surface.

9. The transducer device of claim 4, wherein a difference between the first distance and the second distance is approximately an odd multiple of one quarter of a wavelength of one of the acoustic waves.

10. The transducer device of claim 1, wherein the at least one second reflective surface includes a plurality of second reflective surfaces each approximately equal in width.

11. The transducer device of claim 10, wherein the at least one first reflective surface includes a plurality of first reflective surfaces each having a width approximately equal to the width of each second reflective surface.

12. The transducer device of claim 3, wherein each of the plurality of grooves extends along an entire length of the backing layer.

13. The transducer device of claim 1, wherein the transducer device is incorporated in an ultrasound imaging system.

14. The transducer device of claim 13, wherein the transducer device is positioned in a catheter of the ultrasound imaging system.

15. The transducer device of claim 4, wherein the first distance is less than a distance sufficient to fully attenuate the acoustic waves.

16. A transducer device comprising:
an active layer having a proximal surface; and
a backing layer having a distal side and a proximal side, the distal side being adjacent to the proximal surface;
wherein the proximal side of the backing layer includes (1) one or more first reflective surfaces and (2) one or more second reflective surfaces, the first and second reflective surfaces being approximately parallel to the proximal surface;
wherein the first reflective surfaces are recessed a first distance from the second reflective surfaces; and
wherein the first reflective surfaces and the second reflective surfaces are the only surfaces included on the proximal side that are approximately parallel to the proximal surface, and a surface area of the first reflective surfaces is approximately equal to a surface area of the second reflective surfaces.

17. The transducer device of claim 16, wherein each of the first reflective surfaces and each of the second reflective surfaces extends across substantially all of the proximal side.

18. The transducer device of claim 16, wherein the active layer includes a piezoelectric material that emits acoustic waves in response to application of an electric voltage across the active layer.

19. The transducer device of claim 18, wherein the first distance and the second distance are such that acoustic waves at a center frequency reflected from the first reflective surfaces are approximately a half wavelength out of phase with acoustic waves reflected from the second reflective surfaces.

20. An ultrasound imaging system comprising:
a catheter configured and arranged for insertion into a living being, the catheter having a distal end and a proximal end; and
an imaging device on the catheter, the imaging device configured for imaging interior portions of the living being;
wherein the imaging device includes a plurality of transducer devices, each of the transducer devices including:
an active layer having an proximal surface; and
a backing layer having a distal side and a proximal side, the distal side being adjacent to the proximal surface;
wherein the proximal side includes (1) at least one first reflective surface approximately parallel to the proximal surface and positioned a first distance from the proximal surface, and (2) at least one second reflective surface approximately parallel to the proximal surface and positioned a second distance from the proximal surface, the second distance being different than the first distance;
wherein the at least one first reflective surface has a first area, and the at least one second reflective surface has a second area approximately equal to the first area, and the sum of the first area and the second area is approximately equal to an area of all surfaces of the proximal side that are approximately parallel to the proximal surface.
